(19) United States
(12) Patent Application Publication
(10) Pub. No.: US 2024/0336665 A1
(43) Pub. Date: Oct. 10, 2024

(54) FUSION PROTEINS COMPRISING 072 CORE PEPTIDE AND USE THEREOF

(71) Applicant: Acroimmune Guangzhou Biotech Ltd, Guangzhou (CN)

(72) Inventors: Qunmin ZHOU, Guangzhou (CN); Xianfeng FANG, Guangzhou (CN); Dongling LI, Guangzhou (CN); Libing MU, Guangzhou (CN)

(73) Assignee: Acroimmune Guangzhou Biotech Ltd, Guangzhou (CN)

(21) Appl. No.: 18/708,113

(22) PCT Filed: Nov. 7, 2022
(86) PCT No.: PCT/CN2022/130186

§ 371 Date: May 7, 2024

(30) Foreign Application Priority Data
Nov. 8, 2021 (WO) PCT/CN2021/129220

(51) Int. Cl. C07K 14/47; A61K 38/00; A61P 37/06

(52) U.S. Cl. CPC C07K 14/4727; A61P 37/06; A61K 38/00; C07K 2319/30

(57) ABSTRACT

Provided are compositions of proteins based on fusion of one or more copy of the core peptide to the Fc fragment of human immunoglobulin and their use in treating diseases propagated by inflammations associated with tissue injuries.

### BACKGROUND OF THE INVENTION

Inflammation is an innate immune response to foreign pathogen infection and self-tissue injury. The inducers of inflammation thus can be classified into two categories. The first and the perhaps more potent one is so called as pathogen-associated molecular pattern (PAMP), and the second and less studied one is so called as damage (danger)-associated molecular pattern (DAMP) (Janeway C A Jr. Cold Spring Harbor Symposia on Quantitative Biology. 1989:54:1: Matzinger P. Annual Review of Immunology. 1994; 12:991).

PAMP present on almost all microbial pathogens, and the survival of multi-cellular organisms is dependent on their ability to recognize these PAMP in invading microbial pathogens and to induce immune response or defense reactions (Janeway C A Jr, Medzhitov R. Innate immune recognition. Annu Rev Immunol. 2002; 20:197-216). Some examples of the well characterized PAMPs are Lipopolysaccharide (LPS), Poly (I: C), Pam3Cys, CpG DNA and etc. An evolutionarily ancient family of Toll-like receptors (TLRs) plays a crucial role in the detection of PAMPs in microbial infection and the induction of immune and inflammatory response (Medzhitov R, Preston-Hurlburt P, Janeway C A Jr. A human homologue of the Drosophila Toll protein signals activation of adaptive immunity. Nature. 1997; 388:394; Medzhitov R, Janeway C A Jr. The Toll receptor family and microbial recognition. Trends Microbiol. 2000; 8:452-6).

Unlike PAMP, which are present only on invading microbial pathogens, DAMP by nature, are host self-components which are released by necrotic or damage cells/organs when they are under stress or face foreign microbial invasion. Some of the well characterized DAMP include a variety range of molecules such as the heat-shock proteins (HSP60, HSP70, HSP90), cellular DNA/RNA, RNA chaperone protein such as cold-inducible RNA-binding protein (eCIRP), high mobility group box-1 protein (HMGB1) and so on.

HMGB1, normally a nuclear located chromatin-binding protein, can be actively released by innate immune cells in response to pathogen-derived molecules or passively released by damaged cell in the absence of invasion. By expression surface receptors such as TLRs, RAGE, Tim-3, TREM-1 (triggering receptor expressed on myeloid cells-1), and etc., host innate immune cells can rapidly detect the presence of DAMPs such as HMGB1 and mount inflammatory or inflammation response to resolve the damage.

Meanwhile, in nature, hosts also have developed other defense systems or balance mechanisms to count-act or attenuate the over-immune reaction that might be induced by microbial pathogen infection and/or cellular DAMPs. For instance, pathogens such as bacteria or viruses entering the airway are first trapped by airway surface fluid and epithelial cells and removed from the lung by mucociliary clearance.

One family member with this nature defense function is called mucin. Mucins are highly glycosylated proteins of a large molecular mass (>200 kDa) that are widely expressed on the apical surface of most secretory epithelial cells, particularly in the respiratory, digestive, genitourinary (GI) and endometrial, all of which are exposed to the external environmental stresses. Based on their structure, mucins can be classified into transmembrane/membrane-bound mucins (MUC1, MUC3A, MUC3B, MUC4, MUC12, MUC16, MUC17 and etc.) or secreted/gel-forming mucins (MUC2, MUCSAC, MUC5B, MUC6, MUC19 and etc.). One of the best-characterized mucin molecules is MUC1, which is expressed on the apical surface of a variety of epithelial cells, including the mammary glands, respiratory, gastrointestinal, and reproductive tracts. In the respiratory tract, for example, MUC1 is thought to serve as a physicochemical barrier against inhaled physical-chemical particles, or pathogens such as Pseudomonas aeruginosa. Numerous studies revealed that MUC1 can suppress inflammatory responses induced by microbial pathogens via TLR-dependent or independent signaling pathways.

A key feature of MUC1 or other mucin molecules is that they have a variable number of nearly perfect tandem repeats (TR) and/or adjacent imperfect repeats in the extracellular region. The MUC1 tandem repeat is a 20-amino acid long peptide that is found to be repeated in humans between 20 and more than 120 times. The amino acid sequence of this 20-amino acid core peptide is rich in serine(S), threonline (T) and proline (P) residues (also called as STP motif), and this sequence combination is response for its highly O-linked glycosylation/sialylation status.

In addition to the well-known mucin molecules, some of other so called mucin-like molecules also play an important role in host nature defense against microbial pathogens or DAMPs induced cell damage. One such type of mucin-like molecules is CD24, which is a small glycosylphosphatidylinositol (GPI)-linked glycoprotein widely expressed by epithelial cells and immune cells.

Glycosylated/sialylated MUC1 or mucin-like molecules such as CD24 have been found to be able to bind to HMGB1 and Siglec molecules (Liu Y, Chen G Y and Zheng P: Sialoside-based pattern recognitions discriminating infections from tissue injuries. Curr Opin Immunol. 2011:41-5). Siglecs, stand for sialic acid-binding immunoglobulin-like lectins, are type I transmembrane proteins with a sialic acid binding, IgV-like domain in the extracellular region and immunoreceptor tyrosine-based inhibitory motif (ITIM) or ITIM-like region in their intracellular domains in most Siglecs. ITIM or ITIM-like motifs recruit the phosphatases such as SHP-1, SHP2, and which in turn, leads to a chain of reaction to attenuate or suppress immune response. Siglec-binding molecules or proteins such as soluble CD24 or CD24Fc have been shown to diminish the overall inflammatory response induced by DAMP and found to have therapeutic effects in a number of diseases models including graft vs host diseases (GVHD), rheumatoid arthritis, and pathological setting in which infections cause tissue injuries such as COVID-19, influenza pneumonia and sepsis. Nevertheless, there is still an urgent need to develop a safe and more potent biological product with properties of superior Siglec-binding and enhanced anti-inflammation activities.

### SUMMARY OF THE INVENTION

The present disclosure provides compositions of a glycosylated and/or sialylated core peptide (named as AI-072 core) and compositions of proteins based on fusion of the core peptide to the Fc fragment of human immunoglobulin and their use in treating diseases propagated by inflammations associated with tissue injuries.

The present disclosure provides a protein, comprising one or more copies of a first fragment and one or more copies of a second fragment, each of said first fragment comprises a sequence independently selected from those as set forth in SEQ ID NO: 2 to 4, and each of said second fragment comprises a sequence independently selected from those as set forth in SEQ ID NO: 5 and 8 to 21.

The present disclosure provides an immunoconjugate, comprising the protein of the present disclosure.

The present disclosure provides a nucleic acid, encoding the protein of the present disclosure.

The present disclosure provides a vector, comprising the nucleic acid the present disclosure.

The present disclosure provides a cell, comprising and/or expressing the protein of the present disclosure, the immunoconjugate the present disclosure, the nucleic acid the present disclosure, and/or the vector the present disclosure.

The present disclosure provides a composition, comprising the protein of the present disclosure, the immunoconjugate the present disclosure, the nucleic acid the present disclosure, the vector the present disclosure, and/or the cell the present disclosure, and optionally a pharmaceutically acceptable carrier.

The present disclosure provides a method for preparing or the protein of the present disclosure, comprising culturing the cell the present disclosure under a condition enabling the expression of said AI-072 core fragment or said protein.

The present disclosure provides a method for regulating a Siglec related signaling, comprising administering to a subject in need thereof an effective amount of the protein of the present disclosure, the immunoconjugate the present disclosure, the nucleic acid the present disclosure, the vector the present disclosure, the cell the present disclosure, and/or the composition the present disclosure.

The present disclosure provides a method for regulating an immune response, comprising administering to a subject in need thereof an effective amount of the protein of the present disclosure, the immunoconjugate the present disclosure, the nucleic acid the present disclosure, the vector the present disclosure, the cell the present disclosure, and/or the composition the present disclosure.

The present disclosure provides a method for repressing an immune-mediated tissue damage mediated by danger-associated molecular patterns (DAMPs), comprising administering to a subject in need thereof an effective amount of the protein of the present disclosure, the immunoconjugate the present disclosure, the nucleic acid the present disclosure, the vector the present disclosure, the cell the present disclosure, and/or the composition the present disclosure.

The present disclosure provides a method for preventing, ameliorating and/or treating a disease or condition caused by an inflammatory response arising from tissue injuries from infectious agents, comprising administering to a subject in need thereof an effective amount of the protein of the present disclosure, the immunoconjugate the present disclosure, the nucleic acid the present disclosure, the vector the present disclosure, the cell the present disclosure, and/or the composition the present disclosure.

The present disclosure provides a method for preventing, ameliorating and/or treating a disease or condition caused by acute tissue damage from wound, comprising administering to a subject in need thereof an effective amount of the protein of the present disclosure, the immunoconjugate the present disclosure, the nucleic acid the present disclosure, the vector the present disclosure, the cell the present disclosure, and/or the composition the present disclosure.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates the amino acid sequence within the tandem repeats of human Muc1.

FIG. 2 illustrates the amino acid sequence within the tandem repeats of Muc1 from different species.

FIG. 3 illustrates the schematic structure of the AI-072 fusion protein, comprising AI-072 core peptide and IgG-Fc. The preferred formation of AI-072 fusion protein is a dimer, covalently linked via disulfate chains of the hinge region and non-covalent interactions between CH2 and CH3 domains of human IgG1.

FIGS. 4A-4B illustrate the SDS-PAGE and SEC-HPLC analysis results of the purified AI-072 fusion protein. FIG. 4A illustrates SDS-PAGE gel of the purified AI-072 fusion protein. Two μg of purified AI-072 fusion protein, either in reducing (R) or non-reducing conditions (NR), was loaded into SDS-PAGE gel. After electrophoresis, the gel was stained with Coomassie Brilliant Blue dye. The molecule weight (kDa) of protein marker (M) in the gel was indicated to the left. FIG. 4B illustrates a size exclusion chromatography (SEC)-high performance liquid chromatography (HPLC) separation profile of the purified AI-072 fusion protein.

FIG. 5 illustrates the binding of AI-072 fusion protein to an anti-human Mucin1 mAb SM3 in ELISA. A 96-well plate pre-coated with mAb SM3 was incubated with the binding buffer containing a two-fold serial dilution of AI-072 fusion protein (starting at 10 ug/mL), the bound AI-072 fusion protein was then detected by adding HRP-labeled goat anti-human IgG Fc antibody and o-phenylenediamine (OPD) substrate.

FIG. 6 illustrates the binding of AI-072 protein or CD24Fc protein to human Siglec-10 in ELISA. A 96-well plate pre-coated with HEK293 cell derived recombinant human Siglec-10-mIgGFc protein (at concentration of 200 ng/ml) was incubated with the binding buffer containing a two-fold serial dilution of either AI-072 or CD24Fc fusion protein (all starting at 1.5 mg/ml), the bound AI-072 or CD24Fc fusion protein was detected by adding HRP-labeled goat anti-human IgG Fc antibody and tetramethylbenzidine (TMB) substrate. The EC50 values of the binding are shown in the underneath table.

FIG. 7 illustrates the binding of AI-072 protein or CD24Fc protein to human HMGB1 in ELISA. A 96-well plate pre-coated with recombinant human HMGB1-His tag protein (at concentration of 200 ng/ml) was incubated with the binding buffer containing a two-fold serial dilution of either AI-072 or CD24Fc fusion protein (both starting at 1.5 mg/mL), the bound AI-072 or CD24Fc protein was then detected by HRP-labeled goat anti-human IgG-Fc antibody and TMB substrate. The EC50 values of the binding are shown in the underneath table.

FIGS. 8A-8B illustrate the association of AI-072 protein with human HMGB1 in a pull-down assay. Recombinant HMGB1-His protein sample was incubated with AI-072, human IgG-Fc control, or none for 5 min, then add protein A-conjugated beads to the mixture to capture (or pull-down) the bound proteins. The captured proteins were then separated in an SDS-PAGE gel and visualized by Coomassie Brilliant Blue dye staining. The left gel (A) shows the input samples, and the right gel (B) shows the pull-down samples, as marked. As indicated on the top of each gel, lane 1 represents the sample containing HMGB1 only, lane 2 represents the sample containing HMGB1 and human IgG-Fc, and lane 3 represents the sample containing HMGB1 and AI-072, lane M is the protein molecule weight marker sample. The positions of AI-072 protein, HMGB1 protein in input or pull-down sample are indicated to the right side of each gel, whereas the molecule weight (kDa) of the protein marker is displayed to the left side of each gel.

FIGS. 9A-9D illustrate the therapeutic effects of AI-072 to DSS-induced inflammatory bowel diseases. FIG. 9A illustrates the procedure of DSS-induced mouse inflammatory bowel diseases and the treatment schedule. The AI-072 protein or vehicle control was administrated to model mice by i.p injection on day 0 and day 6. Model mice were then observed daily, with body weight recorded, and survival rate calculated until day 14. FIG. 9B illustrates the animal body change (gram) vs time (day) curve in AI-072 protein treated group or vehicle control treated group. FIG. 9C illustrates the body weight loss rate (%) vs time (day) curve in AI-072 protein treated group or vehicle control treated group. FIG. 9D illustrates the animal survival rate (%) vs time (day) curve in AI-072 protein treated group (n=10) or vehicle control treated group (n=10).

FIGS. 10A-10B illustrate the methods of testing activities of AI-072 to collagen antibody induced arthritis (CAIA). FIG. 10A illustrates the methods of inducing CAIA model and the treatment schedule. Arthritis was induced by i.v. injecting mice with anti-collagen cocktail antibodies (a mixture of 5-clones, 1.5 mg/mouse) on day 0, and followed by i.v. injecting 50 μg LPS on day 3 and day 4. Mice were then randomly separated into two groups, receiving either AI-072 protein (50 mg/kg) or vehicle control on day 0. On day 14, each mouse was re-administered 0.8 mg of anti-collagen cocktail mAbs by i.v. injection, followed by i.p. injection of 35 μg LPS on day 16. On day 19, these mice were treated with either a second dose (1 mg) of AI-072 or saline control. Mice were monitored daily, with body weight recorded, disease scored and survival rate calculated up to day 48. FIG. 10B illustrates the disease score ratio (day/day 19) change starting from day 19 to day 48 in the animal group treated with either AI-072 protein (n=10) or vehicle control (n−10).

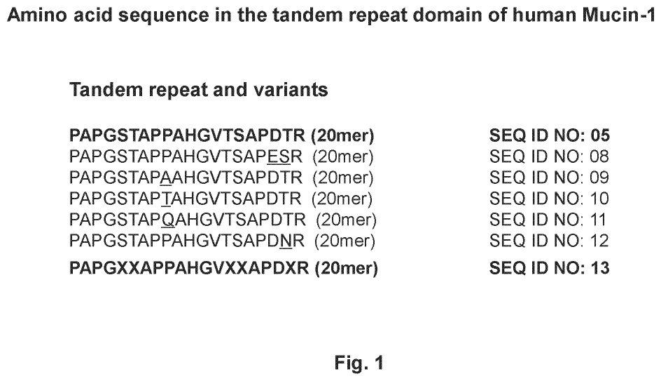

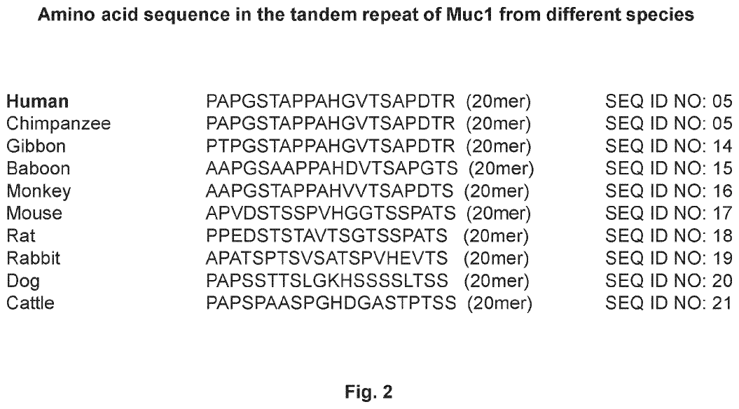

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The term “peptide” or “polypeptide” may refer to a linked sequence of amino acids and may be natural, synthetic, or a modification or combination of natural and synthetic.

The term “glycopeptide” or “glycoprotein” may refer to a modification of natural or synthetic peptide or protein with sugar or oligosaccharide attached or linked to the amino acid residues.

The term “Substantially identical” may refer to a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids.

“Treatment” or “treating,” when referring to protection of an animal from a disease, may refer to preventing, suppressing, repressing, or completely eliminating the disease. Preventing the disease may involve administering a composition of the present invention to an animal prior to onset of the disease. Suppressing the disease may involve administering a composition of the present invention to an animal after induction of the disease but before its clinical appearance. Repressing the disease may involve administering a composition of the present invention to an animal after clinical appearance of the disease.

A “variant” may refer to a peptide or polypeptide that differs in amino acid sequence by the insertion, deletion, or conservative substitution of amino acids, but retain at least one biological activity. Representative examples of “biological activity” may include the ability to bind to a toll-like receptor and to be bound by a specific antibody. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) may be recognized in the art as typically involving a minor change. These minor changes can be identified, in part, by considering the hydropathic index of amino acids, as understood in the art. The hydropathic index of an amino acid may be based on a consideration of its hydrophobicity and charge. It may be known in the art that amino acids of similar hydropathic indexes can be substituted and still retain protein function. In one aspect, amino acids having hydropathic indexes of +2 may be substituted. The hydrophilicity of amino acids can also be used to reveal substitutions that would result in proteins retaining biological function. A consideration of the hydrophilicity of amino acids in the context of a peptide may permit calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions may be performed with amino acids having hydrophilicity values within +2 of each other. Both the hydrophobicity index and the hydrophilicity value of amino acids may be influenced by the particular side chain of that amino acid. Consistent with that observation, amino acid substitutions that are compatible with biological function may be understood to depend on the relative similarity of the amino acids, and particularly the side chains of those amino acids, as revealed by the hydrophobicity, hydrophilicity, charge, size, and other properties.

The term “Mucin” or “Muc” may refer to a protein or peptide. As used herein, Mucin may be a transmembrane/membrane-bound protein. Mucin may encompass Mucin proteins, protein fragments, protein analogs, oligopeptides, and/or a variant thereof. For example, the Mucin fragment may not include the full-length Mucin protein. For example, the Mucin may comprise MUC1, MUC3A, MUC3B, MUC4, MUC12, MUC16, MUC17 and etc. The UniProt No. for MUC1 may be P15941.

The term “fusion” as used herein refers to a fused molecule. wherein the components of the fusion molecule may be linked to each other by bonds, like peptide bonds, either directly or via a peptide linker. The individual peptide chains of the fusion molecule may be linked non-covalently, for example by disulfide bonds.

AI-072 core or fragment

In the present disclosure, a series of recombinant polypeptides or fusion proteins are created, described with their amino acid sequence disclosed here.

For example, the protein of the present disclosure might comprise one or more copies of a first fragment (Fragment A) and one or more copies of a second fragment (Fragment B), each of said first fragment comprises a sequence independently selected from those as set forth in SEQ ID NO: 2 to 4, and each of said second fragment comprises a sequence independently selected from those as set forth in SEQ ID NO: 5 and 8 to 21.

For example, the protein of the present disclosure might comprise 1, 2, 3, 4, 5, 6, 7, 8, 9, 10 or more copies of said first fragment. For example, the protein of the present disclosure might comprise 1, 2, 3, 4, 5, 6, 7, 8, 9, 10 or more copies of said second fragment.

For example, one copy of the first fragment might be fused directly or indirectly to one or more copies of the second fragment. For example, one copy of the first fragment might be fused directly or indirectly to 1, 2, 3, 4, 5, 6, 7, 8, 9, 10 or more copies of the second fragment.

For example, the C-terminus of said the first fragment might be fused directly or indirectly to the N-terminus of one or more copies of the second fragment.

For example, the polypeptide or protein of the present disclosure may comprise a 072-core portion or AI-072 core-fragment. The said 072-core portion or said AI-072 core-fragment might comprise one copy of a first fragment and one or more copies of a second fragment. For example, each of said first fragment might comprise a sequence independently selected from those as set forth in SEQ ID NO: 2 to 4, and each of said second fragment might comprise a sequence independently selected from those as set forth in SEQ ID NO: 5 and 8 to 21.

For example, said protein might exclude a fragment derived from the Mucin1, other than said first fragment and said second fragment. For example, the only fragment derived from the Mucin1 might be said first fragment and said second fragment. For example, in the protein, said first fragment and said second fragment may be directly linked.

For example, said first fragment (fragment A) and second fragment (fragment B) fused indirectly may mean that said fragment A and fragment B might be fused via a linker. For example, the linker may be a (GnS)n linker such as GGGGS, or GGGGSGGGGSGGGGS.

For example, said 072-core portion or said AI-072 core-fragment might be a sequence as set forth in SEQ ID NO: 1.

The amino acid sequence of one of these recombinant polypeptides named as AI-072-core peptide may be shown in SEQ ID NO: 1. This AI-072-core peptide may be composed of a short 11-mer peptide AHDVTSAPDNK (SEQ ID NO: 2) at the N-terminal and one or more tandem repeats with the consensus sequence PAPGSTAPPAHGVTSAPDTR (SEQ ID NO: 05) at the C-terminal. This AI-072-core peptide may contain multiple potential mucin-like O-glycosylation sites (serine or threonine in STP motif) and may be heavily glycosylated and/or sialylated when attached to other partner proteins such as human IgG-Fc and expressed in mammalian cells.

In the present disclosure, variants or modifications of the AI-072 core peptide are also provided. These variants or modifications may be occurred either in the N-terminal short 9-mer or 11-mer peptide region or in the tandem repeat region at the C-terminal. For examples, these variants may have one of the following amino-acid sequences in the short 9-mer or 11-mer peptide at the N-terminal region: APKPATVVT (SEQ ID NO: 03); GSGHASSTP (SEQ ID NO: 04) and/or may have one of the following amino-acid sequences in the tandem repeat region: PAPGSTAPPAHGVTSAPESR (SEQ ID NO: 08); PAPGSTAPAAHGVTSAPDTR (SEQ ID NO: 09); PAPGSTAPTAHGVTSAPDTR (SEQ ID NO: 10); PAPGSTAPQAHGVTSAPDTR (SEQ ID NO: 11); PAPGSTAPPAHGVTSAPDNR (SEQ ID NO: 12) and etc. Each one of these tandem repeats may contain 5 or 6 mucin-like O-glycosylation sites and might be heavily glycosylated and/or sialylated when one or multiple copies of it linked together and fused to other partner proteins such as human IgG-Fc and expressed in mammalian cells.

In the present disclosure, variants or modifications of the AI-072 core peptide may also have such amino-acid sequence PAPGXXAPPAHGVXXAPDXR (X=S or T, SEQ ID NO: 13) in the tandem repeat. The tandem repeat may contain 5 mucin-like O-glycosylation sites and might be heavily glycosylated and/or sialyated when one or multiple copies of it linked together and fused to other partner proteins such as human IgG-Fc and expressed in mammalian cell. Repeats of this 20-amino-acid long peptide such as 2, 3, 4, 5 or more copies of it may have even greater O-glycosylations. Wherein said the core region may be 80% identical to AI-072 core. Wherein said the core region is 80%, 85%, 90%, 91%, 92%, 93%, 94%, 95%, 96%, 97%, 98%, or 99% identical to AI-072 core.

In the present disclosure, some other type variants of the AI-072 core peptide are also provided. These variants may have one of the following amino-acid sequences in the tandem repeat region at the C-terminal: PTPGSTAPPAHGVTSAPDTR (SEQ ID NO: 14) from Gibbon Muc1; AAPGSAAPPAHDVTSAPGTS (SEQ ID NO: 15) from Baboon Muc1; AAPGSTAPPAHVVTSAPDTS (SEQ ID NO: 16) from monkey (Macaca fascicularis) Muc1; APVDSTSSPVHGGTSSPATS (SEQ ID NO: 17) from mouse Muc1, PPEDSTSTAVTSGTSSPATS (SEQ ID NO: 18) from rat Muc1, APATSPTSVSATSPVHEVTS (SEQ ID NO: 19) from rabbit Muc1, PAPSSTTSLGKHSSSSLTSS (SEQ ID NO: 20) from dog Muc1, and PAPSPAASPGHDGASTPTSS (SEQ ID NO: 21) from cattle Muc1. Each one of these tandem repeats may contain at least 5 or more mucin-like O-glycosylation sites and might be heavily glycosylated or sialylated when one or multiple copies of it linked together and fused to other partner proteins such as Fc of human IgG and expressed in mammalian cells.

In this patent application, a series of AI-072 fusion proteins can be created in such a way that 1-, 2-, 3- or more tandem repeats of a peptide PAPGSTAPPAHGVTSAPDTR (SEQ ID NO: 05) are fused with either a short 9-mer peptide APKPATVVT (SEQ ID NO: 03) or GSGHASSTP (SEQ ID NO: 04) or a 11-mer peptide AHDVTSAPDNK (SEQ ID NO: 02) at the N-terminal region and linked with the human IgG-Fc tail at the C-terminal. The human IgG-Fc may consist of hinge-CH2-CH3 regions with the amino acid sequences shown as in SEQ ID NO: 28. The hinge region of the human Fc may come from IgG1, IgG4, IgA1 and have one of the amino acid sequences as set in SEQ ID NO: 22, SEQ ID NO: 23, SEQ ID NO: 24, SEQ ID NO: 25, SEQ ID NO: 26, or SEQ ID NO: 27. For example, said hinge region may refer to a flexible amino acid stretch in the central part of the heavy chains of the immunoglobulin classes. For example, the immunoglobulin may be IgG and IgA.

The present disclosure provides an isolated AI-072 fusion protein, wherein the amino acid sequence of said AI-072 fusion protein may consist of the amino acid sequence as set forth in SEQ ID NO: 30.

The present disclosure provides a protein comprising AI-072 core derived-region, said AI-072 core derived-region may consist of: single copy of AI-072 core fragment; two or more copies of AI-072 core fragment directly or indirectly linked to each other; the amino acid sequence of said AI-072 core fragment may consist of the amino acid sequence as set forth in SEQ ID NO: 01.

For one example of the protein, wherein said two or more AI-072 core fragments may comprise 2, 3, 4, 5 or more of said AI-072 core fragments.

For one example of the protein, wherein at least two of said two or more AI-072 core fragments may be directly linked to each other. For example, the AI-072-core-fragments may be independently directly linked or indirectly linked to each other. For example, two AI-072 core-fragments may be directly linked, and these two AI-072 core fragments may be indirectly linked to another AI-072 core fragment. For example, the directly linked may refers to two or more fragments linked by peptide bonds. For example, the indirectly linked may refers to two or more fragments may be linked by peptide linker, like GnS linker.

For one example of the protein, wherein at least two of said two or more AI-072 core fragments may be indirectly linked to each other via a linker. For one example of the protein, wherein said linker may be a peptide linker. For example, the peptide linker may be a (GnS) n linker such as GGGGS, or GGGGSGGGGSGGGGS.

For one example of the protein, further comprising a second portion, said second portion may comprise a half-life extending portion. For one example of the protein, wherein said half-life extending portion may comprise an immunoglobulin fragment. For example, said half-life extending portion may refer to a portion that could be used to lengthen the half-life of proteins. For example, when the half-life extending portion is fused to the protein, the clearance of said protein might be reduced. For example, protein fusion methods for improving the pharmacokinetics of peptides and small proteins may be widely used.

For one example of the protein, wherein said immunoglobulin fragment may comprise a Fc portion of said immunoglobulin. For one example of the protein, further comprising a second portion, said second portion may comprise an immunoglobulin fragment. For one example of the protein, wherein said immunoglobulin fragment may comprise a Fc portion of said immunoglobulin.

For one example of the protein, wherein said immunoglobulin fragment may comprise a hinge region of said immunoglobulin. For one example of the protein, wherein said immunoglobulin fragment may comprise a CH2 domain. For one example of the protein, wherein said immunoglobulin fragment may comprise a CH3 domain. For one example of the protein, wherein said immunoglobulin fragment may comprise a CH4 domain. For example, said immunoglobulin fragment may comprise hinge region and CH2 and CH3 domains of said Ig protein. For example, said Ig may be selected from the group consisting of IgG1, IgG2, IgG3, IgG4, and IgA. For example, said immunoglobulin fragment may comprise hinge region and CH3 and CH4 domains of said Ig protein. For example, said Ig may be IgM. For example, said immunoglobulin fragment may comprise hinge region and CH2, CH3 and CH4 domains of said Ig protein. For one example of the protein, wherein said immunoglobulin may be selected from the group consisting of IgG1, IgG2, IgG3, IgG4, IgM and IgA.

For one example of the protein, wherein said immunoglobulin may comprise a sequence selected from those as set forth in SEQ ID NO: 22 to 29.

For one example of the protein, wherein said second portion may be directly or indirectly linked to said 072 core-derived-region.

For one example of the protein, wherein said second portion may be indirectly linked to said 072 core-derived-region via a linker. For example, said second portion may be directly linked to said 072 core-derived-region. For example, said second portion may be directly linked to said 072 core-derived-region, and said second portion may not comprise hinge region. For example, said second portion may be directly linked to said AI-072 core derived-region, and said second portion may comprise CH2 and CH3 domains of said Ig protein. For example, said second portion may be directly linked to said AI-072 core-derived-region, and said second portion may comprise CH3 and CH4 domains of said Ig protein.

For one example of the protein, wherein said linker may be a peptide linker.

For one example of the protein, wherein said AI-072 core-derived-region may be linked directly or indirectly to the N-terminus of said second portion.

For one example of the protein, comprising the amino acid sequence as set forth in SEQ ID NO: 05, SEQ ID NO: 06, SEQ ID NO: 07 or SEQ ID NO: 30.

For one example of the protein, which may be a fusion protein.

For one example of the AI-072 core-fragment or the protein, which may be glycosylated.

For one example of the AI-072 core-fragment or the protein, which may be capable of binding to one or more Siglecs.

For one example of the AI-072 core-fragment or the protein, wherein said one or more Siglecs may comprise human Siglec.

For one example of the AI-072 core-fragment or the protein, wherein said one or more Siglecs may comprise Siglec-10.

For one example of the AI-072 core-fragment or the protein, which may be capable of binding to High Mobility Group Protein B1 (HMGB1).

For one example of the AI-072 core-fragment or the protein, wherein said AI-072 core may be derived from human protein. For example, said AI-072 core from other mammalian species are also provided.

The present disclosure provides an immunoconjugate, comprising the AI-072 core-fragment of the present disclosure, or the protein of the present disclosure.

The present disclosure provides a nucleic acid, encoding the AI-072 core-fragment of the present disclosure, or the protein of the present disclosure.

The present disclosure provides a vector, comprising the nucleic acid of the present disclosure.

The present disclosure provides a cell, comprising and/or expressing the AI-072 core-fragment of the present disclosure, the protein of the present disclosure, the immunoconjugate of the present disclosure, the nucleic acid of the present disclosure, and/or the vector of the present disclosure.

The present disclosure provides a composition, comprising the AI-072 core-fragment of the present disclosure, the protein of the present disclosure, the immunoconjugate of the present disclosure, the nucleic acid of the present disclosure, the vector of the present disclosure, and/or the cell of the present disclosure, and optionally a pharmaceutically acceptable carrier.

The present disclosure provides a method for preparing the AI-072 core-fragment of the present disclosure, or the protein of the present disclosure, comprising culturing the cell of the present disclosure under a condition enabling the expression of said AI-072-core-fragment or said protein.

The present disclosure provides a method for regulating a Siglec related signaling, comprising administering to a subject in need thereof an effective amount of the AI-072 core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure. For example, the Siglec related signaling may comprise Siglec-mediated regulation of immune cell function. For example, the Siglec related signaling may comprise CD24-Siglec 10/G interaction. The method of the present disclosure, which may activate the Siglec related signaling. The method of the present disclosure, which may inhibit the Siglec related signaling.

The present disclosure provides the AI-072 core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure, for use in regulating a Siglec related signaling. For example, the Siglec related signaling may comprise Siglec-mediated regulation of immune cell function. For example, the Siglec related signaling may comprise CD24-Siglec 10/G interaction. For example, activating the Siglec related signaling. For example, inhibiting the Siglec related signaling.

The present disclosure provides a use of the AI-072 core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure in the preparation of a medicament, wherein said medicament is used for regulating a Siglec related signaling. For example, the Siglec related signaling may comprise Siglec-mediated regulation of immune cell function. For example, the Siglec related signaling may comprise CD24-Siglec 10/G interaction. For example, activating the Siglec related signaling. For example, inhibiting the Siglec related signaling.

The present disclosure provides a method for regulating an immune response, comprising administering to a subject in need thereof an effective amount of the AI-072 core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure.

The present disclosure provides the AI-072 core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure, for use in regulating an immune response.

The present disclosure provides a use of the AI-072-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure in the preparation of a medicament, wherein said medicament is used for regulating an immune response.

The present disclosure provides a method for repressing an immune-mediated tissue damage mediated by danger-associated molecular patterns (DAMPs), comprising administering to a subject in need thereof an effective amount of the AI-072-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure. For one example of the method, wherein said immune-mediated tissue damage may be selected from the group consisting of graft vs host diseases, immunotherapy-related adverse events, rheumatoid arthritis, inflammatory bowel diseases (IBD), and multiple sclerosis (MS).

The present disclosure provides the AI-072 core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure, for use in repressing an immune-mediated tissue damage mediated by danger-associated molecular patterns (DAMPs). For example, wherein said immune-mediated tissue damage may be selected from the group consisting of graft vs host diseases, immunotherapy-related adverse events, rheumatoid arthritis, inflammatory bowel diseases (IBD), and multiple sclerosis (MS).

The present disclosure provides a use of the AI-072-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure in the preparation of a medicament, wherein said medicament is used for repressing an immune-mediated tissue damage mediated by danger-associated molecular patterns (DAMPs). For example, wherein said immune-mediated tissue damage may be selected from the group consisting of graft vs host diseases, immunotherapy-related adverse events, rheumatoid arthritis, inflammatory bowel diseases (IBD), and multiple sclerosis (MS).

The present disclosure provides a method for preventing, ameliorating and/or treating a disease or condition caused by an inflammatory response arising from tissue injuries from infectious agents, comprising administering to a subject in need thereof an effective amount of the 072-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure. For one example of the method, wherein said disease or condition may be associated with viral infection. For one example of the method, wherein said disease or condition may be COVID-19. For one example of the method, wherein said disease or condition may be influenza. For one example of the method, wherein said disease or condition may be acquired immunodeficiency syndrome (AIDS). For one example of the method, wherein said disease or condition may be associated with bacterial infection. For one example of the method, wherein said disease or condition may be bacterial pneumonia.

The present disclosure provides the protein of the present disclosure, the immunoconjugate of the present disclosure, the nucleic acid of the present disclosure, the vector of the present disclosure, the cell of the present disclosure, and/or the composition of the present disclosure, for use in preventing, ameliorating and/or treating a disease or condition caused by an inflammatory response arising from tissue injuries from infectious agents. For example, wherein said disease or condition may be associated with viral infection. For example, wherein said disease or condition may be COVID-19. For example, wherein said disease or condition may be influenza. For example, wherein said disease or condition may be acquired immunodeficiency syndrome (AIDS). For example, wherein said disease or condition may be associated with bacterial infection. For example, wherein said disease or condition may be bacterial pneumonia.

The present disclosure provides a use of the protein of the present disclosure, the immunoconjugate of the present disclosure, the nucleic acid of the present disclosure, the vector of the present disclosure, the cell of the present disclosure, and/or the composition of the present disclosure in the preparation of a medicament, wherein said medicament is used for preventing, ameliorating and/or treating a disease or condition caused by an inflammatory response arising from tissue injuries from infectious agents. For example, wherein said disease or condition may be associated with viral infection. For example, wherein said disease or condition may be COVID-19. For example, wherein said disease or condition may be influenza. For example, wherein said disease or condition may be acquired immunodeficiency syndrome (AIDS). For example, wherein said disease or condition may be associated with bacterial infection. For example, wherein said disease or condition may be bacterial pneumonia.

The present disclosure provides a method for preventing, ameliorating and/or treating a disease or condition caused by acute tissue damage from wound, comprising administering to a subject in need thereof an effective amount of the AI-072-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure. For one example of the method, wherein said disease or condition may be septicemia, crush syndrome and/or ischemia reperfusion injury.

The present disclosure provides the AI-072-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure, for use in preventing, ameliorating and/or treating a disease or condition caused by acute tissue damage from wound. For example, wherein said disease or condition may be septicemia, crush syndrome and/or ischemia reperfusion injury.

The present disclosure provides a use of the AI-072-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure in the preparation of a medicament, wherein said medicament is used for preventing, ameliorating and/or treating a disease or condition caused by acute tissue damage from wound. For example, wherein said disease or condition may be septicemia, crush syndrome and/or ischemia reperfusion injury.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1

Generation of AI-072 Fusion Protein with Multiple Tandem Repeats from Human Muc1

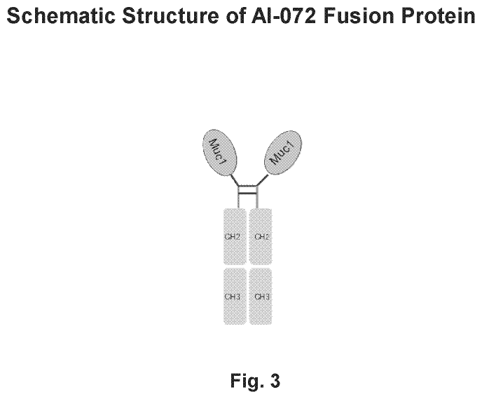

In the present disclosure, a series of recombinant polypeptides or fusion proteins such as AI-072-Fc fusion protein can be generated by using recombinant DNA methods. FIG. 3 shows the schematic structure of one of these AI-072-Fc series fusion proteins (hereafter also just called as AI-072-Fc or AI-072). The amino acid sequence of one particular formation of this fusion protein, AI-072-hIgG1Fc (AI-072-Fc), is shown in SEQ ID NO: 30. The AI-072 core peptide in this fusion protein is composed of a short 11-mer peptide AHDVTSAPDNK (SEQ ID NO: 2) at the N-terminal and tandem repeats of the 20-mer peptide PAPGSTAPPAHGVTSAPDTR (SEQ ID NO: 05) at the C-terminal. To create recombinant DNA construct encoding the full-length AI-072-hIgG1Fc fusion protein, a DNA fragment encoding the peptide of the SEQ ID NO: 01 was fused with a DNA fragment encoding a 26-amino acid long CD24 signal peptide MGRAMVARLGLGLLLLALLLPTQIYS (SEQ ID NO: 32) at the N-terminal and a DNA fragment encoding the hinge-CH2-CH3 region of human IgG1 (SEQ ID NO: 28) at the C-terminal. These DNA fragments were synthesized in-vitro and cloned into an expression plasmid vector pDNA3.1 by using stand recombinant DNA techniques. The recombinant plasmids were transferred into mammalian CHO cells by electroporation. After electroporation, cells were cultured in serum-free media for 6-7 days and supernatants were then collected, passed over a column of Protein A resin (MabSelect from GE Healthcare) at a concentration not exceeding 16 g/L of resin (based on ELISA). The Fc-fusion protein bound on the column was eluted and collected by using low pH solution (0.1 M citric acid, pH 3.5). Eluted protein was then re-suspended in 1×PBS (pH=7.4) and stored at either 4° C. or −20° C. until use.

### Example 2

### Characterization of AI-072-Fc Fusion Protein

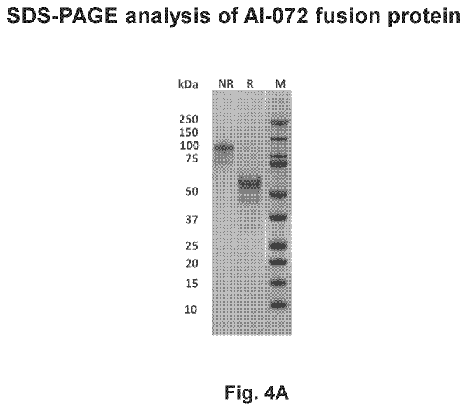

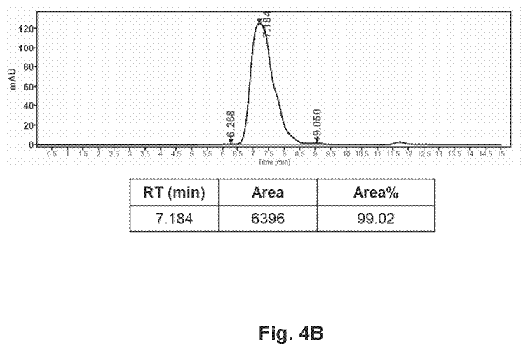

Approximately 2 μg of purified AI-072-Fc fusion protein, either in DTT-reducing or non-reducing conditions was loaded into an SDS-PAGE gel. As shown in FIG. 4A, under DTT-reducing condition, the size of this fusion protein sample is about the half of that in non-reducing condition, this agrees with the natural dimer formation of Fc-fusion protein. Under either DTT-reducing or non-reducing conditions, the appearing molecule weight of AI-072-Fc fusion protein is much greater than that deducted by its protein peptide sequence only. For instance, the monomer of AI-072-Fc mature peptide has 303 amino acid residues, which would have a corresponding protein molecule weight of about 31 kDa. The additional gained molecule weight of about 25 kDa (which contributes almost half of the molecule weight) is most likely from glycans attached to this glycoprotein.

The purified, intact AI-072-Fc protein was further subjected to a SEC-HPLC analysis (FIG. 4B). As shown in FIG. 4B, a main peak with a retention time (RT) at 7.184 min and an area of 99.02% was detected by this SEC-HPLC analysis.

### Example 3

ELISA Analysis of AI-072 Protein with Anti-Human Mucin-1 mAb SM3

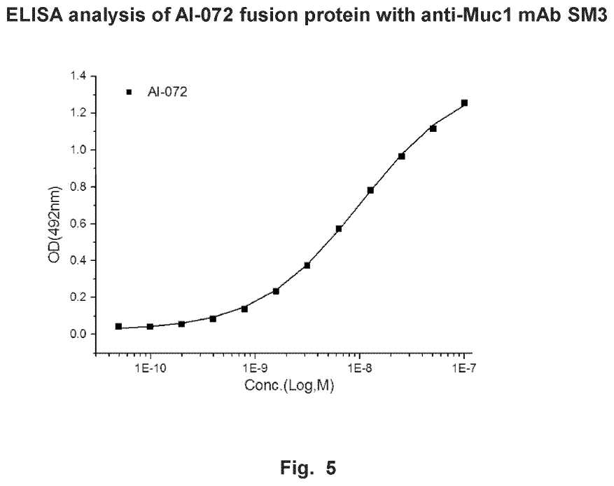

The AI-072-Fe protein, as illustrated in FIG. 3, comprising AI-072 core peptide and human IgG1-Fc region. The AI-072 core contains three tandem repeats of a 20-amino acid long peptide from human Muc1 with the sequence as in SEQ ID NO: 3. Thus, the antigen identity of AI-072-Fc protein can be determined by probing their binding to anti-human Muc1 mAbs such as SM3 (ab22711 from Abcam) in suitable immune assays like an enzyme linked immunosorbent assay (ELISA). For this propose, an antigen binding sandwich ELISA was developed. Briefly, a 96-well plate was coated with 5 μg/mL of SM3 mAb (ab22711 from Abcam) at 4° C. overnight. After blocking with PBS-0.1% Tween20 solution (PBST), 100 μL of two-fold serial dilutions of CHO cell derived AI-072 protein were added into the plate (starting at 10 μg/mL) and the plate was incubated at 37° C. for 1 hour. The plate bound AI-072 protein was detected by adding HRP-labeled goat anti-human IgG-Fc antibody, followed by O-Phenylenediamine (OPD) substrate. After a color development at room temp for 15 mins, 1N HCl stop solution was added into the plate. The OD values at a wavelength of 492 nm (OD492) in each well were then measured. One of the representative ELISA results is showed in FIG. 5. As shown in the Figure, AI-072 protein shows a dose-response binding to SM3 mAb demonstrating that it contains the Muc1 core epitope.

In one embodiment, one may choose 2 or more copies of the 072 core to achieve an even higher O-linked glycosylation/sialylation status.

In another embodiment, one may replace the amino acid sequences within the AI-072 core, either uses homologous amino acid sequences of Muc1 from other species such as chimpanzee, monkey, dog, pig, mouse rat and etc. In yet another embodiment, one may switch the position of serine (Ser or S) or threonine (Thr or T) or increase or alter the pattern of amino acid sequences to increase O-linked glycosylation using known art in the field. In yet another embodiment, one may increase sialyation of the AI-072 core using culture conditions that favors sialylation. In yet another embodiment, one may modify the CHO cells genetically to increase sialyotransferase activity.

### Example 4

### Generation of Super Agonist of Siglec Using Glycosylated/Sialylated AI-072 Core

Defective Siglec function exacerbates inflammation caused by tissues injuries. Diseases associated with such inflammation includes classic sterile inflammation such as drug-induced liver damage, rheumatoid arthritis, inflammatory bowel diseases (IBD), multiple sclerosis, and pathological setting in which infections cause tissue injuries such as COVID-19, influenza pneumonia and sepsis.

Super agonist molecules that show enhanced and broad binding to multiple Siglecs may have therapeutic valuation for treating diseases arising from inflammation caused by tissue injuries.

### AI-072 Protein has a Superior Binding to Siglec-10

To demonstrate that AI-072 protein binds to Siglec-10, an ELSIA assay was developed. In-brief, 96-well plates were coated (100 μL per well) with 0.2 μg/mL of Siglec10-mIgG2aFc fusion protein (AcroBiosystems, SI0-H525b, HEK293 cell derived) at 4° C. overnight. After blocking with SuperBlock (Thermo, 37515) at room temperature for 1 hour, 100 μL of 2-fold serial dilutions of either AI-072 or CD24Fc (all starting at 1.5 mg/ml) were added. The bound AI-072 or CD24Fc protein was then detected by HRP-labeled goat anti-human IgG-Fc antibody (1:5000, Invitrogen, A18829) followed by the addition of Tetramethylbenzidin (TMB) substrate. After a color development at room temp for 15 min, 2N HCl stop solution was added into the plate. The OD values at a wavelength of 450 nm (OD 450 nm) in each well were then measured.

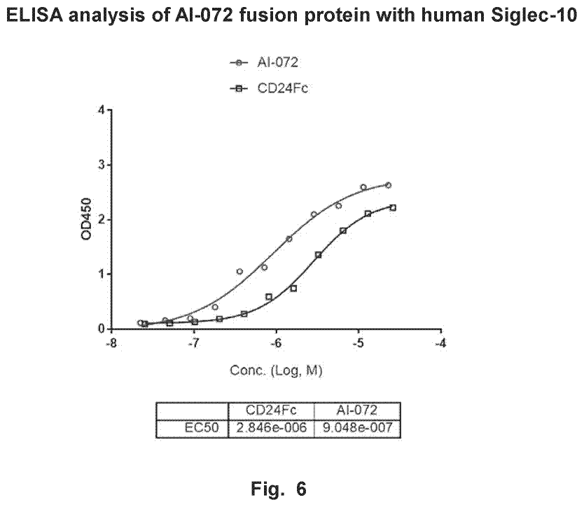

FIG. 6 shows one of the representative ELISA results. As shown in Figure, AI-072 has a higher binding affinity (EC50 at 9.048E-7M) to Siglec10 than CD24Fc has (EC₅₀ at 2.846E-6M). Thus, this data indeed demonstrated that AI-072 has a superior binding to Siglec-10.

### Example 5

### AI-072 Protein has a Superior Binding to High Mobility Group Box 1 (HMGB1)

To demonstrate that AI-072 protein also binds to HMGB1, a similar ELISA assay was developed. In-brief, 96-well plates were coated (100 μL per well) with 0.1 μg/mL HMGB1-His tag protein (AcroBiosystems, HM1-H5220, HEK293 cell derived) at 4° C. overnight. After blocking with SuperBlock (Thermo, 37515) at room temperature for 1 hour, 100 μL of 2-fold serial dilutions of AI-72 or CD24Fc (all starting at 1.5 mg/ml, and diluted in PBST-1% BSA solution containing 1 mM MgCl2 and 1 mM CaCl2) were added. The bound AI-072 or CD24Fc protein was then detected by adding HRP-labeled goat anti-human IgG-Fc antibody (1:1000, Invitrogen, A18829), followed by the addition of tetramethylbenzidin (TMB) substrate. After a color development at room temp for 15 mins, 2N HCl stop solution was then added into the plate. The OD values at a wavelength of 450 nm in each well were then measured.

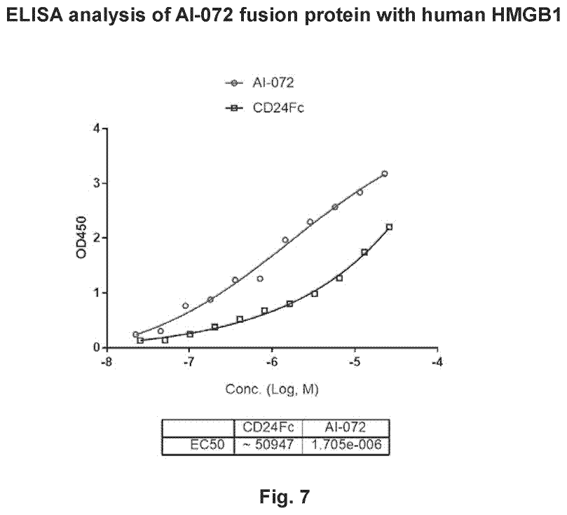

FIG. 7 shows one of the representative ELISA results. As shown in Figure, AI-072 has more than 25-fold higher HMGB1-binding activity (EC50:1.705E-7M) than CD24Fc has (EC₅₀: 5.095E-5M). Thus, this data demonstrated that AI-072 also has a superior binding to HMGB1.

### Probe of AI-072 Protein and HMGB1 Interaction by Pull-Down Assay

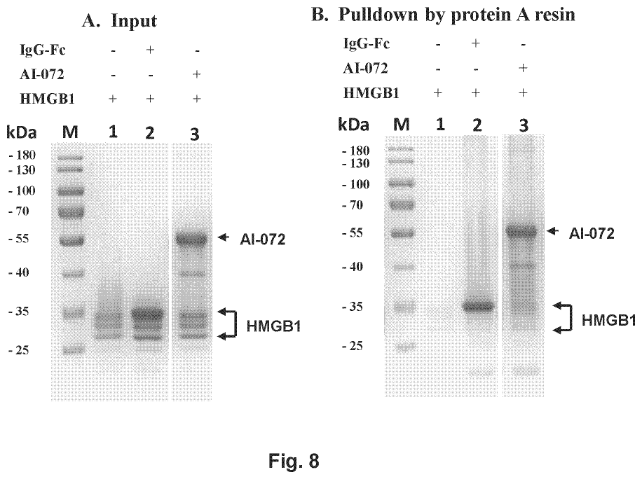

To further verify the superior binding of AI-072 to HMGB1, a protein pull-down assay was developed (FIGS. 8A-8B). In-brief, 8 μL of 500 μg/mL HMGB1-His protein was mixed with either about 3 μg AI-072 or human IgG1-Fc control protein or none, and set at room temp for 5 min. The mixtures were then incubated with protein A-conjugated beads to capture (or pull-down) the bound proteins. The captured proteins were separated in an SDS-PAGE gel and visualized by Coomassie Brilliant Blue dye staining.

FIG. 8B shows one of the representative HMGB1 pull-down assay results. As shown in Fig, HMGB1 proteins are clearly pull-down (captured) by AI-072, but not by IgG1-Fc control protein. Thus, this dada further demonstrated that AI-072 has HMGB1-binding activities.

### Example 6

### AI-072 Protect Mice Against Lethal IBD

### Dextran Sulfate Sodium (DSS)-Induced Inflammatory Bowel Diseases (IBD) in Mice

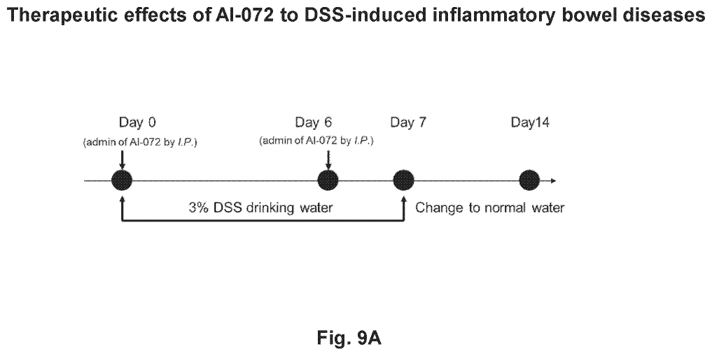

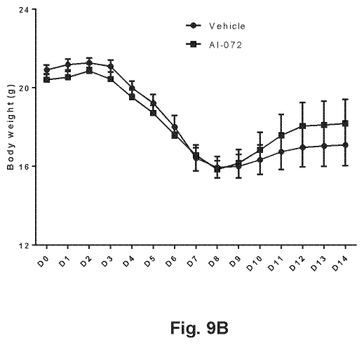

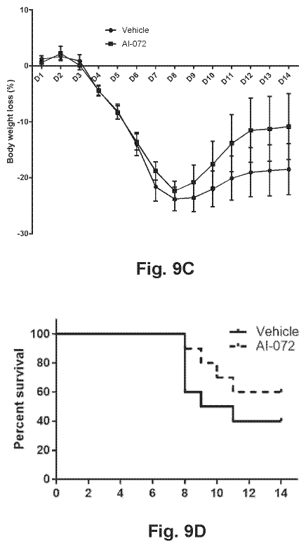

The DSS-induced IBD model is shown in FIG. 9A and the testing results are shown in FIGS. 9B, 9C and 9D. As shown in FIG. 9A and the following table, C57BL/6N mice (6-8 weeks old) were fed with 3% DSS in the drinking water for 7 days, and were monitored daily, for weight loss, disease progression and survival. On day 0, mice were randomly divided into two groups (10 animals/group): one group was administered with AI-072 fusion protein by i.p. injection (dose: 50 mg/kg) on day 0 and day 6, the other group was administered with vehicle control (0.9% NaCl) by same i.p. injection.

| | Modeling | | |
|---|---|---|---|
| Treatment group (N) | agent | Dosage (mg/kg) | Dosing schedule |
| AI-072 (n = 10) | DSS | 50 | Day 0 and Day 6 |
| Vehicle (n − 10) | DSS | 50 | Day 0 and Day 6 |

On day 7, DSS water was removed and mice were then fed with normal drinking water and continue to be monitored daily, for recovery and survival, up to day 14.

The colitis progression was measured by the Disease Activity Index (DAI), and scored as in the following table.

Disease Activity Index (DAI) parameters

| Score | Weight loss | Stool consistency | Bleeding |
|---|---|---|---|
| 0 | No loss | Normal | No blood |
| 1 | 1-5% | Mild soft | Brown color |
| 2 | 5-10% | Very soft | Reddish color |
| 3 | 10-20% | Diarrhea | Bloody stool |
| 4 | >20% | | Gross bleeding |

DAI is obtained by the sum of each individual score.

In this experimental setting, animals in each group started to show signs of disease and weight loss (FIGS. 9B and 9C) from days 3 to 5 after drinking 3% DSS water. Compared to those in the saline treated vehicle control group, animals in AI-072 treated group showed a relatively faster recovery in body weight gain (in grams, FIG. 9B) or a less body weight lose rate (%, FIG. 9C) after switched to the normal drinking water on day 7. As shown in FIG. 9D, at the end of the study (i.e at day 14), the AI-072 treated group had a higher survive rate (60%) than that saline treated vehicle control group (40%), although the difference was not statistically significant (P>0.05, Log-rank test). Nevertheless, these preliminary data indicate that administration of AI-072 protein to subjects might have a partial protection against lethal IBD, such as DSS-induced colitis in mice.

### Example 7

### Diagram for Testing Arthritis

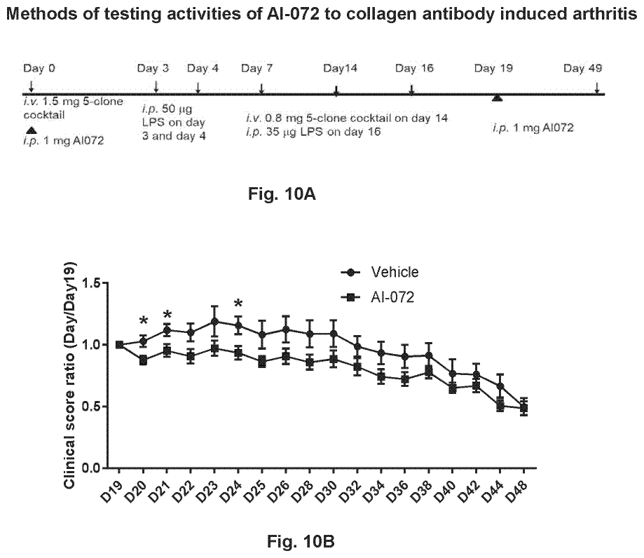

For this propose, a collagen antibody induced arthritis (CAIA) model in mouse was developed. The disease model and treatment schedule are shown in FIG. 10A. Briefly, 7-8 weeks old female Balb/c mice are administered (1.5 mg/mouse) a cocktail of 5 anti-collagen mAbs by i.v. injection on day 0, followed by i.p. injection of 50 μg LPS on day 3 and day 4. On day 0, mice were randomly divided into two different treatment groups (each group has 10 mice): one group was treated with AI-072 (50 mg/kg, i.v. injection), the other was treated with saline vehicle control. On day 14, these mice were re-administered with 0.8 mg of the anti-collagen mAb cocktail by i.v. injection, followed by i.p. injection of 35 μg LPS on day 16. On day 19, AI-072 treated mice were again administrated with a second dose (1 mg) of AI-072 by i.p. injection, whereas control treated mice were administrated with saline by i.p. injection. All of these mice were monitored daily from day 0, up to day 48.

In our experimental setting, animals in each group started to show signs of disease and/or body weight loss from days 5 to day 7 after 1^{st} administration of anti-collagen mAb cocktail. However, as shown in the FIG. 10B, compared to that in vehicle control treated group, AI-072 protein treatment group showed a reduced disease score ratio (day/day 19) from day 20 to day 40, and the mean difference between the AI-072 treated and vehicle treated group is statistically significant on day 20, day 21 and day 24 (P<0.05 by two-tailed T-test, marked as * on the top bar of vehicle group). Therefore, these data demonstrated that AI-072 protein might also have therapeutic value in the treatment of subjects with arthritis.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A protein, comprising
one or more copies of a first fragment and
one or more copies of a second fragment,
wherein each of said first fragment comprises a sequence independently selected from those as set forth in SEQ ID NO: 2 to 4, and
wherein each of said second fragment comprises a sequence independently selected from those as set forth in SEQ ID NO: 5 and 8 to 21, and
wherein said protein excludes a fragment derived from Mucin 1, other than said first fragment and said second fragment.

2. (canceled)

3. The protein of claim 1, wherein one copy of the first fragment is fused directly or indirectly to one or more copies of the second fragment.

4. The protein of claim 1, wherein the C-terminus of said one copy of the first fragment is fused directly or indirectly to the N-terminus of one or more copies of the second fragment.

5. The protein of claim 1, wherein the protein comprises a 072-core portion, and said 072-core portion comprises one copy of a first fragment and one or more copies of a second fragment, each of said first fragment comprises a sequence independently selected from those as set forth in SEQ ID NO: 2 to 4, and each of said second fragment comprises a sequence independently selected from those as set forth in SEQ ID NO: 5 and 8 to 21.

6-9. (canceled)

10. The protein of claim 1, wherein said protein comprise a sequence selected from those as set forth in SEQ ID NO: 1 to 21.

11. The protein of claim 1, wherein said protein comprises a sequence as set forth in SEQ ID NO: 1.

12. The protein of claim 1, further comprising a second portion, said second portion comprises a half-life extending portion.

13-14. (canceled)

15. The protein of claim 1, further comprising a second portion, said second portion comprises an immunoglobulin fragment.

16. The protein of claim 15, wherein said immunoglobulin fragment comprises a Fc portion of said immunoglobulin.

17. (canceled)

18. The protein of claim 15, wherein said immunoglobulin fragment comprises a CH2 domain, a CH3 domain and/or a CH4 domain.

19-21. (canceled)

22. The protein of claim 1, wherein said immunoglobulin comprises a sequence selected from those as set forth in SEQ ID NO: 22 to 29.

23. The protein of claim 12, wherein said second portion is directly or indirectly linked to said 072-core portion.

24-26. (canceled)

27. The protein of claim 1, comprising a sequence selected from those as set forth in SEQ ID NO: 5, 6, 7, 30 and 31.

28. (canceled)

29. The protein of claim 1, which is glycosylated.

30. The protein of claim 1, which is capable of binding to one or more Siglecs.

31-32. (canceled)

33. The protein of claim 1, which is capable of binding to High Mobility Group Protein B1 (HMGB1).

34-35. (canceled)

36. A nucleic acid, encoding the protein of claim 1.

37-38. (canceled)

39. A composition, comprising the protein of claim 1, and optionally a pharmaceutically acceptable carrier.

40. (canceled)

41. A method for regulating a Siglec related signaling, comprising administering to a subject in need thereof an effective amount of the protein of claim 1.

42-43. (canceled)

44. A method for regulating an immune response, comprising administering to a subject in need thereof an effective amount of the protein of claim 1.

45. A method for repressing an immune-mediated tissue damage mediated by danger-associated molecular patterns (DAMPs), comprising administering to a subject in need thereof an effective amount of the protein of claim 1.

46. (canceled)

47. A method for preventing, ameliorating and/or treating a disease or condition caused by an inflammatory response arising from tissue injuries from infectious agents, comprising administering to a subject in need thereof an effective amount of the protein of claim 1.

48-53. (canceled)

54. A method for preventing, ameliorating and/or treating a disease or condition caused by acute tissue damage from wound, comprising administering to a subject in need thereof an effective amount of the protein of claim 1.

55. (canceled)


SEQUENCE LISTING (32 sequences)

SEQ ID NO: 1 - protein, 71 aa, synthetic construct
  REGION 1..71: note = AI-072-core
AHDVTSAPDNKPAPGSTAPPAHGVTSAPDTRPAPGSTAPPAHGVTSAPDTRPAPGSTAPPAHGVTSAPDTR

SEQ ID NO: 2 - protein, 11 aa, synthetic construct
  REGION 1..11: note = Muc1-N1
AHDVTSAPDNK

SEQ ID NO: 3 - protein, 9 aa, synthetic construct
  REGION 1..9: note = Muc1-N2
APKPATVVT

SEQ ID NO: 4 - protein, 9 aa, synthetic construct
  REGION 1..9: note = Muc1-N3
GSGHASSTP

SEQ ID NO: 5 - protein, 20 aa, synthetic construct
  REGION 1..20: note = Muc1-TRx1
PAPGSTAPPAHGVTSAPDTR

SEQ ID NO: 6 - protein, 40 aa, synthetic construct
  REGION 1..40: note = Muc1-TRx2
PAPGSTAPPAHGVTSAPDTRPAPGSTAPPAHGVTSAPDTR

SEQ ID NO: 7 - protein, 60 aa, synthetic construct
  REGION 1..60: note = Muc1-TRx3
PAPGSTAPPAHGVTSAPDTRPAPGSTAPPAHGVTSAPDTRPAPGSTAPPAHGVTSAPDTR

SEQ ID NO: 8 - protein, 20 aa, synthetic construct
  REGION 1..20: note = Muc1-V1
PAPGSTAPPAHGVTSAPESR

SEQ ID NO: 9 - protein, 20 aa, synthetic construct
  REGION 1..20: note = Muc1-V2
PAPGSTAPAAHGVTSAPDTR

SEQ ID NO: 10 - protein, 20 aa, synthetic construct
  REGION 1..20: note = Muc1-V3
PAPGSTAPTAHGVTSAPDTR

SEQ ID NO: 11 - protein, 20 aa, synthetic construct
  REGION 1..20: note = Muc1-V4
PAPGSTAPQAHGVTSAPDTR

SEQ ID NO: 12 - protein, 20 aa, synthetic construct
  REGION 1..20: note = Muc1-V5
PAPGSTAPPAHGVTSAPDNR

SEQ ID NO: 13 - protein, 20 aa, synthetic construct
  REGION 1..20: note = Muc1-V6
  SITE 5: note = misc_feature - Xaa = Ser Thr
  SITE 6: note = misc_feature - Xaa = Ser Thr
  SITE 14: note = misc_feature - Xaa = Ser Thr
  SITE 15: note = misc_feature - Xaa = Ser Thr
  SITE 19: note = misc_feature - Xaa = Ser Thr
PAPGXXAPPAHGVXXAPDXR

SEQ ID NO: 14 - protein, 20 aa, synthetic construct
  REGION 1..20: note = Gibbon-muc1
PTPGSTAPPAHGVTSAPDTR

SEQ ID NO: 15 - protein, 20 aa, synthetic construct
  REGION 1..20: note = Baboon-muc1
AAPGSAAPPAHDVTSAPGTS

SEQ ID NO: 16 - protein, 20 aa, synthetic construct
  REGION 1..20: note = Monkey-muc1
AAPGSTAPPAHVVTSAPDTS

SEQ ID NO: 17 - protein, 20 aa, synthetic construct
  REGION 1..20: note = Mouse-muc1
APVDSTSSPVHGGTSSPATS

SEQ ID NO: 18 - protein, 20 aa, synthetic construct
  REGION 1..20: note = Rat-muc1
PPEDSTSTAVTSGTSSPATS

SEQ ID NO: 19 - protein, 20 aa, synthetic construct
  REGION 1..20: note = Rabbit-muc1
APATSPTSVSATSPVHEVTS

SEQ ID NO: 20 - protein, 20 aa, synthetic construct
  REGION 1..20: note = Dog-muc1
PAPSSTTSLGKHSSSSLTSS

SEQ ID NO: 21 - protein, 20 aa, synthetic construct
  REGION 1..20: note = Cattle-muc1
PAPSPAASPGHDGASTPTSS

SEQ ID NO: 22 - protein, 17 aa, synthetic construct
  REGION 1..17: note = IgG1-hinge 1
EPKSCDKTHTCPPCPAP

SEQ ID NO: 23 - protein, 17 aa, synthetic construct
  REGION 1..17: note = IgG1-hinge 2
EPKSSDKTHTCPPCPAP

SEQ ID NO: 24 - protein, 17 aa, synthetic construct
  REGION 1..17: note = IgG1-hinge 3
EPKSSDKTHTSPPSPAP

SEQ ID NO: 25 - protein, 13 aa, synthetic construct
  REGION 1..13: note = IgG1-hinge 4
DKTHTCPPCPAPE

SEQ ID NO: 26 - protein, 15 aa, synthetic construct
  REGION 1..15: note = IgG4-hinge
ESKYGPPCPPCPAPA

SEQ ID NO: 27 - protein, 18 aa, synthetic construct
  REGION 1..18: note = IgA1-hinge
VPSTPPTSPSTPPTPSPS

SEQ ID NO: 28 - protein, 232 aa, synthetic construct
  REGION 1..232: note = human IgG1 hingeCH2CH3 region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 260 aa, synthetic construct
  REGION 1..260: note = human IgG1Fc protein
MGRAMVARLGLGLLLLALLLPTQIYSGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 303 aa, synthetic construct
  REGION 1..303: note = AI-072-Fc fusion protein
AHDVTSAPDNKPAPGSTAPPAHGVTSAPDTRPAPGSTAPPAHGVTSAPDTRPAPGSTAPPAHGVTSAPDTREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 329 aa, synthetic construct
  REGION 1..329: note = signal peptide+AI072Fc fusion protein
MGRAMVARLGLGLLLLALLLPTQIYSAHDVTSAPDNKPAPGSTAPPAHGVTSAPDTRPAPGSTAPPAHGVTSAPDTRPAPGSTAPPAHGVTSAPDTREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 26 aa, synthetic construct
  REGION 1..26: note = signal peptide-CD24
MGRAMVARLGLGLLLLALLLPTQIYS